(19) United States
(12) Patent Application Publication
(10) Pub. No.: US 2022/0259571 A1
(43) Pub. Date: Aug. 18, 2022

(54) HOST-INDEPENDENT EXPRESSION OF BACTERIOPHAGES

(71) Applicant: TECHNISCHE UNIVERSITÄT MÜNCHEN, München (DE)

(72) Inventors: Kilian VOGELE, Raisting (DE); Friedrich SIMMEL, München (DE); Quirin EMSLANDER, Erding (DE)

(21) Appl. No.: 17/627,854

(22) PCT Filed: Jul. 16, 2020
(86) PCT No.: PCT/EP2020/070178

§ 371 Date: Jan. 18, 2022

(30) Foreign Application Priority Data
Jul. 18, 2019 (EP) 19187078.1

(51) Int. Cl. C12N 7/00

(52) U.S. Cl. CPC C12N 7/00; C12N 2795/10252

(57) ABSTRACT

The present invention relates to a method for producing a bacteriophage in a cell-free host-independent expression system and a corresponding composition comprising a cell lysate of an organism which is different to the host of the bacteriophage, a at least one bacteriophage-host specific factor and a genome of a bacteriophage. The respective kits are also encompassed. The invention moreover refers to a bacteriophage obtained by the method of the invention and uses thereof.

### FIELD OF THE INVENTION

The present invention relates to a method for producing a bacteriophage in a cell-free host-independent expression system and a corresponding composition comprising a cell lysate of an organism which is different to the host of the bacteriophage, at least one bacteriophage-host specific factor and a genome of a bacteriophage. The respective kits are also encompassed. The invention moreover refers to a bacteriophage obtained by the method of the invention and uses thereof.

### BACKGROUND OF THE INVENTION

Bacteriophages are viruses that specifically infect a host bacterium and multiply at the expense of that bacterium. The biotechnological applications of bacteriophages are very broad and range from evolution-based selection methods, such as the evolutionary improvement of the activity of enzymes (Esvelt et al. 2011), to the so-called phage display, which can be used to generate and optimize biological drugs such as therapeutic antibodies (Bazan et al. 2012), to the use of bacteriophages themselves as substitutes for antibiotics in bacteriophage therapy (Barbu et al. 2016). The latter is based on the natural ability of bacteriophages to attack and destroy specifically pathogenic bacteria (lysis). However, the development and production of phage-based therapeutics and diagnostics is still hampered by the difficulty of a simple and safe production method for bacteriophages. Until now, bacteriophages are produced by cultivation with the appropriate bacterium/pathogen (Pirnay et al., 2018). This requires compliance with the appropriate safety regulations for the respective bacteria, as well as the possibility to cultivate them. For dangerous pathogens handling is very difficult and costly due to the need of specially trained personnel in special facilities.

The cell-free synthesis of proteins has a number of advantages over cellular expression, especially when toxic proteins are produced for the bacteria or non-natural amino acids are to be introduced into the proteins. Protein synthesis can be performed with the transcription and translation apparatus of lysed cells. After purification, it is free of host DNA and enables the expression of the desired protein through the external addition of DNA. It is even possible to synthesize several proteins simultaneously or metabolites (Garamella et al. 2016). A number of cell-free expression systems are available, the composition of which can vary greatly. The so-called “PURE System” (Shimizu et al. 2001) consists of purified proteins, while crude cell extract of E. coli contains almost all intracellular proteins, including those that are not necessary for expression (Sun et al. 2013). In such an crude cell extract it has already been shown that it is possible to express infectious wild-type bacteriophages (Shin et al. 2012) as well as proteins (Garamella et al. 2016).

However, not all bacteriophages can easily be produced in an E. coli cell lysate. Hence one is limited to E. coli based phages. Alternatively, the cell extract can also be obtained from other bacterial strains. However, this is connected with very complex screenings to find the suitable conditions to get a high-quality cell extract which can also express bacteriophages. In addition, if the bacteriophages are to be used as drugs, it must be shown that the cell extract is free of toxins and other harmful substances, like prophages. Therefore, there is a need for an efficient, less laborious and general applicable method for the production of bacteriophages which is independent from the host organism of the bacteriophage.

### OBJECTIVES AND SUMMARY OF THE INVENTION

The invention solves this problem by the addition of at least one bacteriophage-host specific factor or nucleotide sequence encoding said factor to the cell lysate that is derived from a microorganism which is not the host of the bacteriophage. Thereby, the bacteriophage can be produced in a standard cell lysate which is derived from a microorganism different to the host of the bacteriophage.

Accordingly, a first aspect of the invention refers to a method for producing a bacteriophage in a cell-free host-independent expression system:
providing a cell lysate derived from a microorganism which is different to the host of the bacteriophage,
adding at least one bacteriophage-host specific expression factor and/or a nucleotide sequence encoding the at least one bacteriophage-host specific expression factor,
adding the genome of a bacteriophage.

In one embodiment, the cell lysate is E. coli cell lysate. E. coli cell lysate is well studied and well characterized, e.g. regarding toxins and other potentially harmful compounds. Thus, using E. coli cell lysate is advantageous in particular for the production of bacteriophages for medical purposes and application in the food sector. In such embodiments the natural host of the bacteriophage to be produced is typically not E. coli: For example the bacteriophage may be phi29 having a natural host which is B. subtilis.

Typically, the bacteriophage-host specific factor is a compound of the host organism of the bacteriophage, such as a molecule (e.g. protein) involved in replication, such as a DNA polymerase binding protein, or transcription, e.g. a transcription factor, and/or a subunit of RNA polymerase II, host factor that facilitates the ass or in the self-assembly of the bacteriophage. In a specific embodiment, the bacteriophage is phi29 and the bacteriophage-host specific factor is sigA. The bacteriophage-host specific expression factor may be an isolated molecule or molecule complex. The bacteriophage-host specific expression factor may be a co-expressed molecule.

The bacteriophage-host specific expression factor may be provided as nucleic acid sequence encoding the isolated factor for co-expression in the cell lysate. This is particularly advantageous for factors which are not or only difficult to isolate and purify due to loss of activity or due to toxicity for the host organism expressing the factor. Co-expression of the factor in the expression system of the invention allows to expedite the production method, since the steps for purifying the factor can be omitted.

The genome of the bacteriophage may be in form of isolated native DNA, synthesized DNA, PCR product of the bacteriophage genome or a Yeast Artificial Chromosome.

In specific embodiments the host is a bacterium or an archaeon, preferably a bacterium. More specifically the host is a gram positive or gram negative bacterium, preferably a gram positive bacterium, such as B. subtilis.

Another aspect of the invention refers to a composition for producing a bacteriophage in a host-independent expression system, comprising
a cell lysate derived from a microorganism which is different to the host of the bacteriophage,
at least one bacteriophage-host specific factor, and
a genome of a bacteriophage.

A further aspect refers to a kit for producing a bacteriophage in a cell free expression system comprising:
a genome of a bacteriophage,
at least one bacteriophage-host specific expression factor, and
optionally a cell lysate of an organism different to the host of the bacteriophage.

A further aspect refers to a bacteriophage obtained by the method as described herein and its use as a medicament. More specifically the said bacteriophage may be used in the prevention or treatment of a bacterial infection in a subject.

Also contemplated the use of the bacteriophage obtained by the method of the invention for avoiding bacterial growth in food or beverage or for detecting specific microorganisms.

### FIGURE LEGENDS

FIG. 1: Schematic diagram showing the required constitutes for the expression of a non-E.coli phages in an E. coli cell-free system, here especially for the Bacillus subtilis phage phi29, where the necessary host factor is sigA, which is encoded on a pET20b(+) plasmid under an T7 promotor.

FIG. 2: Spot-assay of the cell-free reactions with the plasmid encoding for the host-factor sigA (top) and without (right). In the sample with the plasmid encoding sigA lysis of the bacteria occurred and in the sample without the plasmid no lysis occurred.

FIG. 3: Phage titer in plaque forming units per millilitre of the cell-free reactions without the plasmid encoding for the host-factor sigA (left) and with (right).

### DETAILED DESCRIPTION OF THE INVENTION

Before the invention is described in detail with respect to some of its preferred embodiments, the following general definitions are provided.

The present invention as illustratively described in the following may suitably be practiced in the absence of any element or elements, limitation or limitations, not specifically disclosed herein.

The present invention will be described with respect to particular embodiments and with reference to certain figures but the invention is not limited thereto but only by the claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of ±10%, and preferably of ±5%.

Technical terms are used by their common sense. If a specific meaning is conveyed to certain terms, definitions of terms will be given in the following in the context of which the terms are used.

A first aspect of the invention refers to a method for producing a bacteriophage in a cell-free host-independent expression system comprising the steps:
providing a cell lysate derived from a microorganism which is different to the host of the bacteriophage,
adding at least one bacteriophage-host specific expression factor and/or a nucleotide sequence encoding the at least one bacteriophage-host specific expression factor,
adding the genome of a bacteriophage.

A bacteriophage is a virus that infects the microorganisms, namely bacteria or archaea. It is composed of capsid proteins that encapsulate a DNA or RNA genome. After infection of their genome into the cytoplasm, bacteriophages replicate in the microorganism using the transcription and translation apparatus of the microorganism. Phages are classified by the international Committee on Taxonomy of Viruses according to morphology and nucleic acid, including Ackermannviridae, Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Globuloviridae, Inoviridae, Leviviridae, Microviridae, Plasmaviridae, Pleolipoviridae, Portogloboviridae, Spharolipoviridae, Spiraviridae, Tectiviridae, Tristromaviridae, Turriviridae.

The host of the bacteriophage is a microorganism, in particular an archeon or bacterium, which can be infected and in which the bacteriophage can replicate. A bacteriophage may have a single host or a broad host spectrum, i.e. the bacteriophage may be capable of infecting different types of microorganisms. The skilled person is aware of methods for determining whether a microorganism is a host for a bacteriophage, such as the spot test, the plaque test, the routine test dilution (RTD) or the cell culture lysis which are known by the skilled person and for exampled described in Hyman, 2019; Pharmaceuticals 2019, 12, 35.

A microorganism which is different for the host of the bacteriophage, is a microorganism which cannot be infected by the bacteriophage and in which bacteriophage cannot replicate.

The bacteriophages can only replicate in their host organism, therefore, producing bacteriophages in a cell lysate that is derived from a microorganism which is different to the host is not possible. The inventors found that in order to enable the production of bacteriophages in a cell lysate derived from a microorganism which is different from host of the bacteriophage, i.e. host-independent, a bacteriophage-host specific factor has to be added to the cell lysate.

“Cell lysate” as used herein refers to a composition comprising the components of cells of a microorganism, in particular a bacterium, after lysis. The cell lysate is therefore void of intact cells, i.e. cell-free. Typically the cell lysate is free of host DNA. Preferably the cell lysate is free of host DNA and membranes. Moreover, the cell lysate may be free of small metabolites. The cell lysate comprises the transcription and translation machinery of the organism which is different to the host of the bacteriophage.

Preferably the cell lysate is E. coli lysate. In such embodiments the natural host of the bacteriophage is not E. coli. More preferably the cell lysate is E. coli Rosetta™(DE3) cell lysate.

“Bacteriophage-host specific factor” is a molecule of the host of the bacteriophage which is not present in the organism which is different to the host of the bacteriophage. The molecule enables the expression and/or the self-assembly in said “non-host” cell lysate. The factor may be a single molecule or several molecules, e.g. building a complex. Typically, the factor is involved in transcription, i.e. a protein involved in transcription, such as transcription factor, e.g. sigA of B. subtilits, or the subunit of RNA polymerase II. Further examples for bacteriophages and transcription factors are Pseudomonas aeruginosa with rpoD, Klebsiella pneumoniae with SigL, Staphylococcus aureus with sigA, Mycobacterium tuberculosis with sigA, Acinetobacter baumannii with RpoD.

In some embodiments, the bacteriophage-host specific expression factor is an isolated molecule or molecule complex. Alternatively or in addition, the bacteriophage-host specific expression factor is provided as nucleic acid sequence encoding the isolated factor for co-expression in the cell lysate.

Typically, the bacteriophage-host specific factor is a compound of the host organism of the bacteriophage, such as a molecule (e.g. protein) involved in replication, e.g. a DNA polymerase binding protein, or transcription, e.g. a transcription factor, and/or a subunit of RNA polymerase II, host factor that facilitates the ass or in the self-assembly of the bacteriophage. In a specific embodiment, the bacteriophage is phi29 and the bacteriophage-host specific factor is sigA.

The amino acid sequence of sigA is set out below (wherein the star represents a stop codon/end of sequence) and in SEQ ID NO: 1

MADKQTHETELTFDQVKEQLTESGKKRGVLTYEEIAERMSSFEIESDQMD
EYYEFLGEQGVELISENEETEDPNIQQLAKAEEEFDLNDLSVPPGVKIND
PVRMYLKEIGRVNLLSAKEEIAYAQKIEEGDEESKRRLAEANLRLVVSIA
KRYVGRGMLFLDLIQEGNMGLMKAVEKFDYRKGYKFSTYATWWIRQAITR
AIADQARTIRIPVHMVETINKLIRVQRQLLQDLGREPTPEEIAEDMDLTP
EKVREILKIAQEPVSLETPIGEEDDSHLGDFIEDQEATSPSDHAAYELLK
EQLEDVLDTLTDREENVLRLRFGLDDGRTRTLEEVGKVFGVTRERIRQIE
AKALRKLRHPSRSKRLKDFLE*

The bacteriophage-host specific expression factor may be an isolated molecule or molecule complex. The bacteriophage-host specific expression factor may be a co-expressed molecule.

The bacteriophage-host specific expression factor may be identified by comparison of the transcription/translation machinery of the host of the bacteriophage and the microorganism different to the host of the bacteriophage used for the cell lysate.

To determine the missing host factor usually the most promising candidate are the sigma factors, where as the primary sigma factor of the host bacteria is usually the most promising candidate, as they are responsible for the “housekeeping” genes. To choose a sigma factor one has to search for the recognition sequence of the corresponding host factor. Therefore also the recognition sequences of the early genes of the phage can be compared with the recognitions sequences of the host bacteria genome to choose the right sigma factor.

For other host factors which bind to phage proteins a ligand binding assay can be performed, to identify the missing host factor. It is also possible to perform mass spectrometry like isolation of proteins on nascent DNA coupled with mass spectrometry, with labelling the corresponding phage molecules (Reyes et al. 2017).

The inventors found that the addition of a host factor is enough to enable the expression and/or the self-assembly of the bacteriophage in a “non-host” cell lysate, i.e., the host factors endogenously present in the cell lysate, e.g. the sigma-factors of the cells from which the lysate is prepared, surprisingly do not block or interfere with the expression and/or self-assembly of the bacteriophage.

The term “microorganism” refers to a bacterium or an archaeon. Preferably, the microorganism is a bacterium.

The host of the bacteriophage is a microorganism. Preferably, the host is a bacterium. The host may be a gram positive or gram negative bacterium. Exemplary hosts a B. subtilis, Pseudomonas aeruginosa Klebsiella pneumoniae, Staphylococcus aureus, Mycobacterium tuberculosis, Acinetobacter baumannii, Enterobacteriaceae, Enterococcus faecium, Helicobacter pylori, Salmonellae, Neisseria gonorrhoeae, Shigella, Campylobacter, Streptococcus pneumoniae and Haemophilus influenzae. In a specific embodiment the host is B. subtilis.

In some specific embodiments the bacteriophage is a phi29 bacteriophage. The host of phi29 bacteriophage is B. subtilis. E. coli is not a host of phi29 bacteriophage. Thus, the production of phi29 bacteriophage in E. coli cell lysate is only possible if a bacteriophage-host specific expression factor, namely sigA factor, is added to the E. coli lysate. SigA factor is a protein that is produced by the host B. subtilis and is required for the transcription for the genome of the bacteriophage.

Conditions for production of bacteriophages in cell-lysate are described in Rustad et al., 2018, Garamella et al. 2016, Shin 2012).

The genome of the bacteriophage may be provided in form of isolated native DNA, synthesized DNA, a PCR product of the bacteriophage genome or a Yeast Artificial Chromosome. The genome of the bacteriophage may be also parts of the genome, e.g. a gene set that enables the production of the bacteriophage.

Not every bacteriophage genome can be transformed into a host cell. By using cell lysate and a suitable host factor, the present method advantageously allows the modification of the genome of bacteriophages that replicate in hosts that cannot be transformed with a modified bacteriophage genome, such as a synthesized bacteriophage genome, a PCR product of the bacteriophage genome or a Yeast Artificial Chromosome.

The method may further comprise adding small metabolites and/or buffer.

A further aspect of the invention refers to a composition for producing a bacteriophage in a host-independent expression system, comprising
a cell lysate derived from a microorganism which is different to the host of the bacteriophage,
at least one bacteriophage-host specific factor, and
a genome of a bacteriophage.

In such a cell-free extract the bacteriophage can be produced by the use of the transcription and translation machinery of the microorganism from which the extract is derived from supplemented with the bacteriophage-host specific factor.

Another aspect of the invention refers to a kit for producing a bacteriophage in a cell free expression system comprising:
a genome of a bacteriophage,
at least one bacteriophage-host specific expression factor,
optionally cell lysate of an organism different to the host of the bacteriophage.

Moreover the invention refers to a bacteriophage obtained by the method as described herein.

Another aspect of the invention refers to a bacteriophage obtained by the method as described herein. A further aspect of the invention refers to a bacteriophage as described herein for use as a medicament, for example for use in the treatment of a bacterial infection in a subject.

Other aspects of the invention refer to the use of the bacteriophage as described herein for avoiding bacterial growth in food or beverage, agriculture and or for detecting specific microorganisms.

### Methods

### DNA Preparation

Phage DNA was purified from previous prepared Phage stocks form titers above 108 PFU/ml by phenol-chloroform extraction, followed by an ethanol precipitation. The concentration was adjusted to approximately 5 nM, determined by adsorption at 260 nm.

### Cell Extract Preparation

For the generation of crude S30 cell extract a BL21-Rosetta 2(DE3) mid-log phase culture was bead-beaten with 0.1 mm glass beads in a Minilys homogenizer (Peqlab, Germany) as described in by Sun et al. (doi:10.3791/50762) The extract was incubated at 37° C. for 80 min to allow the digestion of genomic DNA, and was then dialyzed for 3 h at 4° C. with a cut-off of 10 kDa (Slide-A-Lyzer Dialysis Cassettes, Thermo Fisher Scientific). Protein concentration was estimated to be 30 mg/mL with a Bradford essay. The composite buffer contained 50 mM Hepes (pH 8), 5.5 mM ATP and GTP, 0.9 mM CTP and UTP, 0.5 mM dNTP, 0.2 mg/mL tRNA, 26 mM coenzyme A, 0.33 mM NAD, 0.75 mM cAMP, 68 mM folinic acid, 1 mM spermidine, 30 mM PEP, 1 mM DTT and 4.5% PEG-8000. As an energy source in this buffer phosphoenolpyruvate (PEP) was utilized instead of 3-phosphoglyceric acid (3-PGA). All components were stored at −80 ° C. before usage. A single cell-free reaction consisted of 42% (v/v) composite buffer, 25% (v/v) DNA plus additives and 33% (v/v) S30 cell extract. For ATP regeneration 13.3 mM maltose, against DNA degradation add 3.75 nM GamS and 1 U of T7 RNA polymerase (NEB, M0251S) were added to the reaction mix.

### Phage Expression

For the phage expression 1 nM of the phage genome was added and 1 nM of the Plasmid encoding encoding sigA regulated with a T7 promotor. The sample is incubated at 29° C. for the duration.

### Results

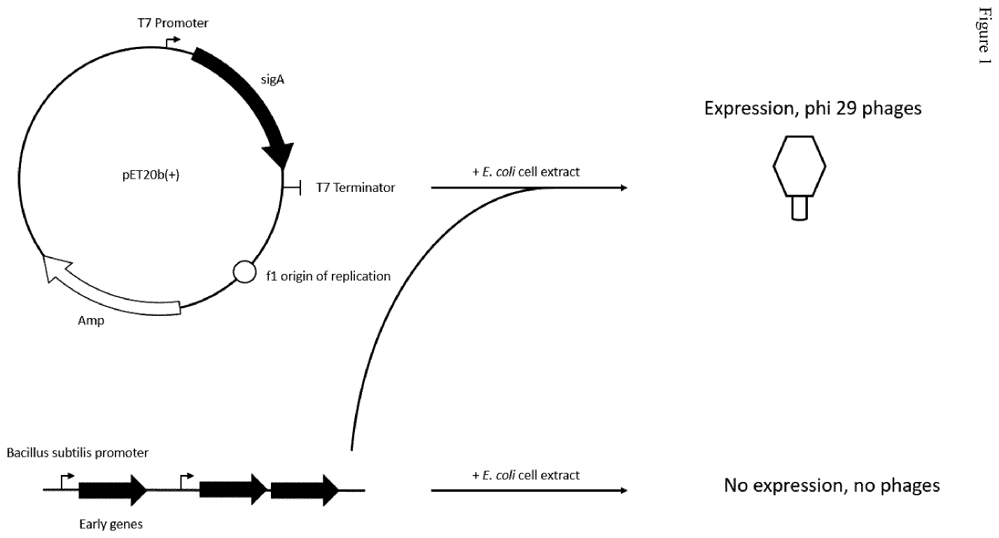

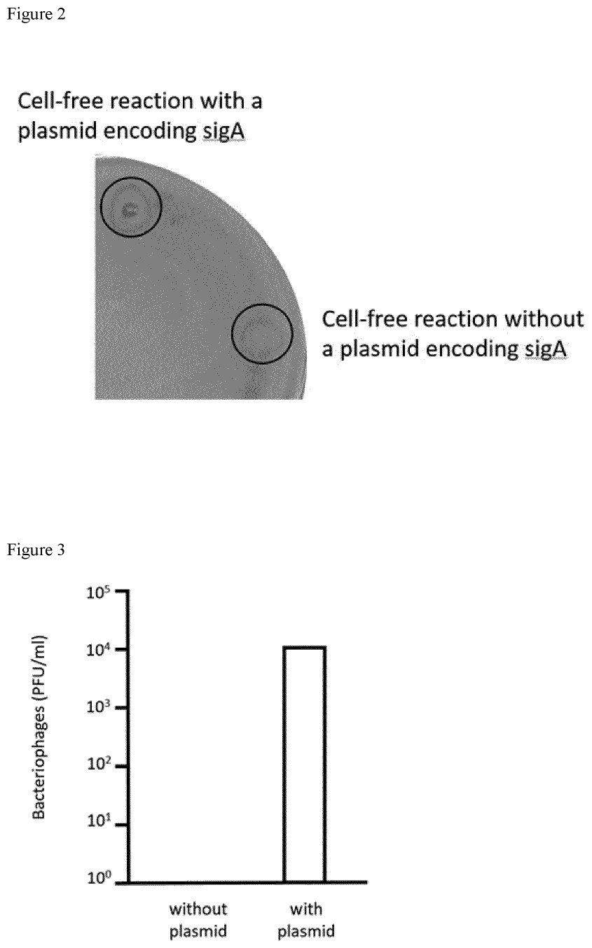

For the host-independent in vitro expression a host factor is required. For the Bacillus subtilis phage phi29, the host factor sigA is required, which is responsible for the “housekeeping genes” of Bacillus subtilis. With this sigma factor the phi29 phage can be expressed in a cell-free expression system derived from E. coli. To provide sigA a plasmid encoding this protein under a T7 Promoter is added to the cell-free reaction mix, beside the phage DNA (FIG. 1). Only if the plasmid and the phage DNA is added to the cell-free system phages were expressed. To proof this, a spot assay was performed, which showed lysis of a lawn of Bacillus subtilis bacteria only if the reaction mix contained the phage DNA, the plasmid encoding the host factor sigA and the cell-free system. In the negative control no lysis of the bacteria was observed (FIG. 2). Beside the spot assay also a plaque assay was performed. From that the concentration of phages was determined in plaque forming units per ml (PFU/ml). In the negative control, without the host factor no phages were detected, whereas in the sample where beside the phage DNA and the cell extract the plasmid encoding for the host-factor was present, 10⁴ PFU/ml were expressed in vitro (FIG. 3).

The application further comprises the following items:
Item 1. Method for producing a bacteriophage in a cell-free host-independent expression system comprising the following steps:
   providing a cell lysate derived from a microorganism which is different to the host of the bacteriophage,
   adding at least one bacteriophage-host specific expression factor and/or a nucleotide sequence encoding the at least one bacteriophage-host specific expression factor,
   adding the genome of a bacteriophage.
Item 2. Method according to item 1, wherein the cell lysate is E. coli cell lysate.
Item 3. Method according to item 1 or 2, wherein the host of the bacteriophage is not E. coli.
Item 4. Method according to any one of the preceding items, wherein the bacteriophage is a phi29 bacteriophage.
Item 5. Method according to any one of the preceding items, wherein the at least one bacteriophage-host specific factor is a compound of the host organism of the bacteriophage.
Item 6. Method according to any one of the preceding items, wherein the at least one bacteriophage-host specific expression factor is a protein involved in transcription.
Item 7. Method according to item 7, wherein the at least one bacteriophage-host specific expression is a transcription factor.
Item 8. Method according to any one of the preceding items, wherein the at least one bacteriophage-host specific expression factor is an isolated molecule or molecule complex.
Item 9. Method according to any one of the preceding items, wherein the at least one bacteriophage-host specific expression factor is provided as nucleic acid sequence encoding the isolated factor for co-expression in the cell lysate.
Item 10. Method according to any one of the preceding items, wherein the at least one bacteriophage-host specific expression factor is sigA.
Item 11. Method according to any one of the preceding items, wherein the genome of the bacteriophage is provided in form of isolated native DNA, synthesized DNA, PCR product of the bacteriophage genome or a Yeast Artificial Chromosome.
Item 12. Method according to any one of the preceding items, wherein the method further comprises adding small metabolites.
Item 13. Method according to any one of the preceding items, wherein the host is a bacterium or an archaeon.
Item 14. Method according to any one of the preceding items, wherein the host is a gram positive or gram negative bacterium.
Item 15. Method according to any one of the preceding items, wherein the host is a gram positive bacterium.
Item 16. Method according to any one of the preceding items, wherein the host is B. subtilis.
Item 17. Composition for producing a bacteriophage in a host-independent expression system, comprising
   cell lysate derived from a microorganism which is different to the host of the bacteriophage,
   at least one bacteriophage-host specific factor, and
   genome of a bacteriophage.
Item 18. Composition for producing a bacteriophage in a host-independent expression system, comprising
   cell lysate derived from a microorganism which is different to the host of the bacteriophage,
   at least one bacteriophage-host specific expression factor, and
   genome of a bacteriophage.
Item 19. Kit for producing a bacteriophage in a cell free expression system comprising:
   Genome of a bacteriophage,
   at least one bacteriophage-host specific expression factor,
   optionally cell lysate of an organism different to the host of the bacteriophage.
Item 20. Bacteriophage obtained by the method according to items 1 to 16.
Item 21. Bacteriophage according to item 20 for use as a medicament.
Item 22. Bacteriophage according to item 20 for use in the prevention or treatment of a bacterial infection in a subject.
Item 23. Use of the bacteriophage according to item 20 for avoiding bacterial growth in food or beverage.
Item 24. Use of the bacteriophage for detecting specific microorganisms.

### REFERENCES

Barbu et al. (2016): Phage Therapy in the Era of Synthetic Biology. In: Cold Spring Harbor perspectives in biology 8 (10).

Bazan et al. (2012): Phage display—a powerful technique for immunotherapy. 1. Introduction and potential of therapeutic applications. In: Human voaccines & immunotherapeutics 8 (12), s. 1817-1828.

Esvelt et al. (2011): A System for the continuous directed evolution of biomolecules. In: Nature 472 (7344), S. 499-503. DOI:10.1038/nature09929.

Garamella et al. (2016): The All E. coli TX-TL Toolbox 2.0: A Platform for Cell-Free Synthetic Biology. In: ACS synthetic biology 5 (4), s. 344-355.

Hyman et al. (2019): Phages for Phage Therapy: Isolation, Characterization, and Host Range Breadth. In: Pharmaceuticals 2019, 12(1), 35

Pirnay, et al. (2018). The magistral phage. Viruses, 10(2), 64.

Shimizu, et al. (2001): Cell-free translation reconstituted with purified components. In: Nature biotechnology 19 (8), S. 751-755.

Shin, et al. (2012): Genome replication, Synthesis, and assembley of the bacteriophage T7 in a single cell-free reaction. In: ACS synthetic biology 1 (9), S. 408-413.

Sun, et al. (2013): Protocols for implementing an Escherichia coli base TX-TL cell-free expression System for synthetic biology. In: Journal of visualized experiments: JoVE (79), e50762.

Reyes et al (2017): Identifying Host Factors Associated with DNA Replicated During Virus Infection. In: Mol Cell Proteomics. 2017 December; 16(12):2079-2097.

Rustad Cell-free TXTL synthesis of infectious bacteriophage T4 in a single test tube reaction Synthetic Biology, Volume 3, Issue 1.

1. Method for producing a bacteriophage in a cell-free host-independent expression system comprising the following steps:
providing a cell lysate derived from a microorganism which is different to the host of the bacteriophage,
adding at least one bacteriophage-host specific expression factor and/or a nucleotide sequence encoding the at least one bacteriophage-host specific expression factor,
adding the genome of a bacteriophage.

2. Method according to claim 1, wherein the cell lysate is E. coli cell lysate.

3. Method according to claim 1 or 2, wherein the host of the bacteriophage is not E. coli.

4. Method according to any one of the preceding claims, wherein the host of the bacteriophage is a gram positive bacterium, preferably B. subtilis.

5. Method according to any one of the preceding claims, wherein the bacteriophage is a phi29 bacteriophage.

6. Method according to any one of the preceding claims, wherein the at least one bacteriophage-host specific expression factor is a transcription factor.

7. Method according to any one of the preceding claims, wherein the at least one bacteriophage-host specific expression factor is sigA.

8. Composition for producing a bacteriophage in a host-independent expression system, comprising
cell lysate derived from a microorganism which is different to the host of the bacteriophage,
at least one bacteriophage-host specific factor, and
genome of a bacteriophage.

9. Kit for producing a bacteriophage in a cell free expression system comprising:
Genome of a bacteriophage,
at least one bacteriophage-host specific expression factor,
optionally cell lysate of an organism different to the host of the bacteriophage.

10. Bacteriophage obtained by the method according to claims 1 to 7.

11. Bacteriophage according to claim 10 for use as a medicament.

12. Bacteriophage according to claim 10 for use in the prevention or treatment of a bacterial infection in a subject.

13. Use of the bacteriophage according to claim 10 for avoiding bacterial growth in food or beverage.

14. Use of the bacteriophage for detecting specific microorganisms.


SEQUENCE LISTING (1 sequence)

SEQ ID NO: 1 - protein, 371 aa, Bacillus subtilis
MADKQTHETELTFDQVKEQLTESGKKRGVLTYEEIAERMSSFEIESDQMDEYYEFLGEQGVELISENEETEDPNIQQLAKAEEEFDLNDLSVPPGVKINDPVRMYLKEIGRVNLLSAKEEIAYAQKIEEGDEESKRRLAEANLRLVVSIAKRYVGRGMLFLDLIQEGNMGLMKAVEKFDYRKGYKFSTYATWWIRQAITRAIADQARTIRIPVHMVETINKLIRVQRQLLQDLGREPTPEEIAEDMDLTPEKVREILKIAQEPVSLETPIGEEDDSHLGDFIEDQEATSPSDHAAYELLKEQLEDVLDTLTDREENVLRLRFGLDDGRTRTLEEVGKVFGVTRERIRQIEAKALRKLRHPSRSKRLKDFLE